(19) United States
(12) Patent Application Publication
(10) Pub. No.: US 2005/0136094 A1
(43) Pub. Date: Jun. 23, 2005

(54) Drug delivery device with mesh based suture tab

(76) Inventors: David Watson, San Jose, CA (US); George Purtell, Westford, MA (US); Brian Levy, Rochester, NY (US); Dominic V. Ruscio, Webster, NY (US); Jay F. Kunzler, Canandaigua, NY (US); Michael M. Schmidt, Cambridge, MA (US); Matthew S. Jonasse, Sodus, NY (US); Joseph C. Salamone, Fairport, NY (US)

Correspondence Address: Bausch & Lomb Incorporated, One Bausch & Lomb Place, Rochester, NY, 14604-2701, US

(21) Appl. No.: 11/012,592

(22) Filed: Dec. 15, 2004

Related U.S. Application Data
(60) Provisional application No. 60/531,880, filed on Dec. 22, 2003.

(51) Int. Cl. A61F 2/00; A61K 9/22

(52) U.S. Cl. 424/427; 604/890.1

(57) ABSTRACT

A drug delivery device for placement in the eye includes a drug core comprising a pharmaceutically active agent, and a holder that holds the drug core. The holder is made of a material impermeable to passage of the active agent and includes an opening for passage of the pharmaceutically agent therethrough to eye tissue. The holder includes a tab, the tab being associated with a biocompatible surgical fabric.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and includes at least one opening for passage of the pharmaceutically active agent therethrough to eye tissue. The device further includes a suture tab having a mesh component to improve the suture tab's mechanical integrity at the suture site, thus minimizing the possibility of suture failure.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye for treating various eye diseases. Examples are found in the following patents, the disclosures of which are incorporated herein by reference: U.S. 2002/0086051A1 (Viscasillas); U.S. 2002/0106395A1 (Brubaker); U.S. 2002/0110591A1 (Brubaker et al.); U.S. 2002/0110592A1 (Brubaker et al.); U.S. 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include an inner drug core including a pharmaceutically active agent and some type of holder for the drug core made of an impermeable material such as silicone or other hydrophobic materials. The holder includes one or more openings for passage of the pharmaceutically active agent therethrough to eye tissue. Many of these devices include at least one layer of material permeable to the active agent, such as polyvinyl alcohol (PVA).

The holder may be associated with a suture tab. The suture tab may be made of a material other than the impermeable material of the holder. The suture tab can be secured to the holder by standard adhesives. When the suture tab is made of a material different than the impermeable material of the holder, the difference in the properties of the materials that make up the holder and the suture tab may result in difficulty in providing effective adhesion between the materials. Making the holder and suture tab of the same material, while improving adhesion, may result in the suture hole failing when it is sutured to the implantation site. For example, if the surgeon uses excessive force while tying the suture knot, failure of the suture hole may occur by the suture tearing through the suture tab material. Therefore, there is a need to provide devices with suture tabs capable of being effectively adhered to the drug holder while still demonstrating mechanical integrity at the suture site.

### BRIEF DESCRIPTION OF THE DRAWINGS

FIG. 1 is a side view of a first embodiment of a drug delivery device of this invention.

FIG. 2 is a top plan view of the device of FIG. 1.

### SUMMARY OF THE INVENTION

According to a first embodiment, this invention relates to a drug delivery device for placement in the eye, comprising: a drug core comprising a pharmaceutically active agent; and a holder that holds the drug core, the holder being made of a material impermeable to passage of the active agent and including at least one opening for passage of the pharmaceutically active agent therethrough to eye tissue, wherein the device optionally includes a suture tab attached to the holder, the suture tab being associated with a biocompatible surgical fabric.

The invention further comprises a method for making the drug delivery device. The method of the invention comprises forming openings in a wall of a holder with a laser; and inserting in the holder a drug core comprising a pharmaceutically active agent; wherein the holder is made of a material impermeable to passage of the active agent and including a tab, the tab being associated with a biocompatible surgical fabric. Of the invention is an assembly containing the device for packaging and shipping. In one embodiment, the assembly comprises an upper surface; a first flange extending upwardly from the upper surface and defining a containment region for containing the device, said containment region including a support surface for supporting the device in the containment region; a second flange extending upwardly from the upper surface, said second flange surrounding the first flange and including an upper flange surface for sealing of lidstock thereto; and at least one side wall extending downwardly from the upper surface and serving to support the package on a work surface, further comprising a recess extending below the device support surface in the containment region, wherein the first flange comprises two protrusions extending upwardly from the upper surface and defining the containment region, and the recess has the form of an elongated groove separating the two protrusions and extending transversely to the containment region, wherein the two protrusions are arcuate, wherein the maximum width between inner surfaces of an individual protrusion is 10 mm.

### DETAILED DESCRIPTION OF PREFERRED EMBODIMENTS

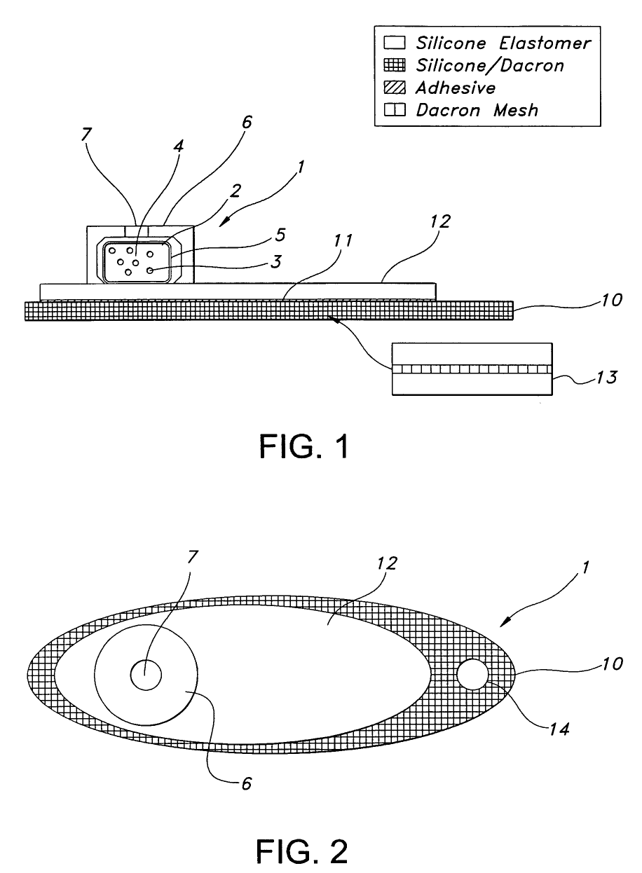

FIGS. 1 and 2 illustrate a first embodiment of a device of this invention. Device 1 is a sustained release drug delivery device for implanting in the eye. Device 1 includes inner drug core 2 including a pharmaceutically active agent 3.

This active agent 3 may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecamil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins; enzyme inhibitors; antioxidants; anticataract drugs; aldose reductase inhibitors; cytoprotectants; cytokines, cytokine inhibitors, and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent employed is fluocininolone acetonide.

In addition to the illustrated materials, a wide variety of materials may be used to construct the devices of the present invention. The only requirements are that they are inert; non-immunogenic and of the desired permeability. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

As shown in the illustrated embodiment, active agent 3 may be mixed with a matrix material 4. Preferably, matrix material 4 is a polymeric material that is compatible with body fluids and the eye. Additionally, matrix material should be permeable to passage of the active agent 3 therethrough, particularly when the device is exposed to body fluids. For the illustrated embodiment, the matrix material is PVA. Also, in this embodiment, inner drug core 2 may be coated with a coating 5 of additional matrix material that may be the same or different from material 4 mixed with the active agent. For the illustrated embodiment, the coating 5 employed is also PVA.

Materials suitable as coating 5 would include materials that are non-bioerodible and are permeable or can be made to be permeable to the active agent. Preferably, the coating material will be release rate limiting. Suitable polymers, depending upon the specific active agent, would include polyvinyl alcohol, ethylene vinyl acetate, silicone, polylactic acid, nylon, polypropylene, polycarbonate, cellulose, cellulose acetate, polyglycolic acid, polylactic glycolic acid, cellulose esters or polyether sulfone. Coating 5 may also be any of the various semipermeable membrane-forming compositions or polymers such as those described in U.S. Patent Publication No. 2002/0197316 (hereby incorporated by reference). Coating 5 may also include plasticizer and pharmaceutically acceptable surfactant such as those described in U.S. patent Publication No. 2002/0197316.

Further examples of semipermeable polymers that may be useful according to the invention herein can be found in U.S. Pat. No. 4,285,987 (hereby incorporated by reference), as well as the selectively permeable polymers formed by the coprecipitation of a polycation and a polyanion as described in U.S. Pat. Nos. 3,541,005; 3,541,006 and 3,546,142 (hereby incorporated by reference).

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, active agent passes through any permeable matrix material 4 and permeable or semi-permeable coating 5, and exits the device through passageway 7. For the illustrated embodiment, the holder is made of silicone, especially polydimethylsiloxane (PDMS) material.

A wide variety of materials may be used to construct the devices of the present invention. The only requirements are that they are inert, non-immunogenic and of the desired permeability. Materials that may be suitable for fabricating the device include naturally occurring or synthetic materials that are biologically compatible with body fluids and body tissues, and essentially insoluble in the body fluids with which the material will come in contact.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

The illustrated embodiment includes a tab 12 which may be made of a wide variety of materials, including those mentioned above for the matrix material and/or the holder. Tab 12 is provided in order to attach the device to a desired location in the eye, for example, by suturing. For the illustrated embodiment, tab 12 is made of PDMS material and is adhered to the inner drug core 2 with adhesive (not shown). Adhesive may be a curable silicone adhesive, a curable polyvinyl alcohol (PVA) solution, or the like.

Tab 12 is associated with biocompatible surgical fabric 10, for example, Dacron mesh, to improve the mechanical integrity of the tab at the suture site. As more fully disclosed hereinafter, the biocompatible surgical fabric 10 can be operatively associated with tab 12. The fabric can be woven, knit or nonwoven and be manufactured from nonbioabsorbable materials. Nonbioabsorbable surgical fabrics include those that are fabricated from such polymers as polyethylene, polypropylene, nylon, polyethylene terephthalate, polytetrafluoroethylene, polyvinylidene fluoride, and the like. A wide variety of materials may be used to construct the biocompatible surgical fabric of the devices of the present invention. The only requirements are that they are inert; non-immunogenic and of the desired mechanical strength. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids is to be avoided since dissolution of the fabric could affect the integrity of the suture tab. It may be possible to use soluble fabrics when the fabric is substantially surrounded with an insoluble material such as PDMS material.

The biocompatible surgical fabric or mesh is associated with the material of the tab using methods known to those of skill in the art. For example, the surgical fabric can be attached to the tab with silicone based adhesive 11. Alternatively, the tab can be coextruded with the surgical fabric or molded around the surgical fabric. Other methods of associating the suture tab with the surgical fabric would include sandwich construction in which the surgical fabric is placed between films of the tab material.

An embodiment of the invention herein may be prepared as follows. Polyester reinforced silicone sheeting is obtained from a commercial supplier (Specialty Silicone Fabricators, Paso Robles, Calif.). The reinforced silicone sheeting is then cut into the desired shape for the suture tab (for example, by laser). The suture tab is removed from the backing sheet and thoroughly cleaned (for example with three successive rinses with isopropyl alcohol for a minimum of 2 hours each).

The tablets are placed into the arrays according to procedures known to those of skill in the art. The tablets and array are then cured.

The array of tablets is placed onto a support, for example a die plate. The array and support are then placed onto an assembly plate. Because certain adhesives can strongly adhere to surfaces such as bare stainless steel, a coated assembly plate is preferable. The coating should be durable and greatly reduce the adherence of the adhesive to the plate. An example of a suitable coating is available under the trade name NEDOX from General Magnaplate, Linden, N.J.

The individual cups of the array are then punched into the existing wells of the assembly plate. Under vacuum, a thin film of adhesive, for example RTV silicon adhesive, is applied to the entire surface of a cup and tablet. One reinforced suture tab is then placed onto a cup using the assembly plate as a guide for alignment. This step is then repeated for all cups of the array. An even pressure is then applied across the surface of the assembly plate, for example with a 4 mm diameter stainless steel rod. The pressure is applied to expel all excess adhesive and ensure that the suture tabs are flush to the surface of the assembly plate. The surface of the assembly plate is then wiped to remove all excess adhesive.

The assembly devices are allowed to dry for 72 hours or other suitable length of time. After drying, each implant is removed from the assembly plate. The finished devices are then inspected and packaged for use or storage.

According to preferred embodiments, the holder is also extracted to remove residual materials therefrom. For example, in the case of silicone, the holder may include lower molecular weight materials such as unreacted monomeric material and oligomers. It is believed that the presence of such residual materials may also deleteriously affect adherence of the holder surfaces. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder may be plasma treated, following extraction, in order to increase the wettability of the holder and improve adherence of the drug core and/or the tab to the holder. Such plasma treatment employs oxidation plasma in an atmosphere composed of an oxidizing media such as oxygen or nitrogen containing compounds: ammonia, an aminoalkane, air, water, peroxide, oxygen gas, methanol, acetone, alkylamines, and the like, or appropriate mixtures thereof including inert gases such as argon. Examples of mixed media include oxygen/argon or hydrogen/methanol. Typically, the plasma treatment is conducted in a closed chamber at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes.

A cylindrical cup of silicone b is separately formed, for example by molding, having a size generally corresponding to the tablet and a shape as generally shown in FIG. 2. This silicone holder is then extracted with a solvent such as isopropanol. An opening 7 is placed in the silicone holder, for example, with a laser. If desired, a drop of liquid PVA may be placed into the holder through the opening 7 of the holder. Optionally, a preformed PVA plug such as a PVA film may be placed into the holder to aid in the control of the delivery of the active agent. Then, the inner drug core tablet is placed into the silicone holder through the same opening 7 and pressed into the cylindrical holder. If the drop of liquid PVA has been applied, the pressing of the tablet causes the liquid PVA to fill the space between the tablet inner core and the silicone holder, thus forming permeable layer 5 shown in FIG. 1. A layer of adhesive is applied to the open side of the holder to fully enclose the inner drug core tablet at this end. Tab 12 is inserted at this end of the device. Heating the assembly cures the liquid PVA and adhesive.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

It should be understood that the preferred device comprises a suture tab. However, a suture tab is not necessary for therapeutic operation of the device.

The device is typically provided to the end user in a sealed sterilized package, for example, by gamma irradiation, for example, such as is disclosed in U.S. patent application Ser. No. 10/183,804, the contents of which are incorporated by reference herein.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

1. A drug delivery device for placement in the eye, comprising:
a drug core comprising a pharmaceutically active agent; and
a holder that holds the drug core, the holder being made of a material impermeable to passage of the active agent and including a tab,
the tab being associated with a biocompatible surgical fabric.

2. The device of claim 1, wherein the impermeable material comprises silicone.

3. The device of claim 1, wherein the tab is adhered to at least one of the drug core and the holder.

4. The device of claim 1, wherein the tab is molded integrally with the holder.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the biocompatible surgical fabric is selected from the group consisting of woven, knit and non-woven nonbioabsorbable surgical materials.

8. The device of claim 1, wherein the drug core is cylindrical.

9. The device of claim 1, wherein the drug core is coated with a material permeable to said active agent.

10. The device of claim 1, comprising a mixture of pharmaceutically active agents.

11. A method of making a drug delivery device for attachment to eye tissue, comprising:
forming openings in a wall of a holder with a laser; and
inserting in the holder a drug core comprising a pharmaceutically active agent;
wherein the holder is made of a material impermeable to passage of the active agent and including a tab,
the tab being associated with a biocompatible surgical fabric.

12. The method of claim 11, wherein the impermeable material comprises silicone.

13. The method of claim 11, wherein a tab is adhered to at least one of the drug core and the holder.

14. The method of claim 11, wherein a tab molded integrally with the holder.

15. The method of claim 11, wherein the drug core comprises a mixture of the active agent and a matrix material permeable to said active agent.

16. The method of claim 15, wherein the matrix material comprises polyvinyl alcohol.

17. The method of claim 11, wherein the holder comprises a cylinder that surrounds the drug core, and an end of the cylinder includes the openings.

18. The method of claim 11, wherein the drug core is cylindrical.

19. The method of claim 11, wherein the drug core is coated with a material permeable to said active agent.

20. The method of claim 11, comprising a mixture of pharmaceutically active agents.

21. A package for storing an implantable medical device during storage and shipping, comprising:
an upper surface;
a first flange extending upwardly from the upper surface and defining a containment region for containing the device, said containment region including a support surface for supporting the device in the containment region;
a second flange extending upwardly from the upper surface, said second flange surrounding the first flange and including an upper flange surface for sealing of lidstock thereto; and
at least one side wall extending downwardly from the upper surface and serving to support the package on a work surface,
further comprising a recess extending below the device support surface in the containment region,
wherein the first flange comprises two protrusions extending upwardly from the upper surface and defining the containment region, and the recess has the form of an elongated groove separating the two protrusions and extending transversely to the containment region,
wherein the two protrusions are arcuate, wherein the maximum width between inner surfaces of an individual protrusion is 10 mm, and
wherein the implantable medical device is the device of claim 1.

22. The package of claim 21 wherein the implantable medical device is the device of claim 2.

23. The package of claim 21 wherein the implantable medical device is the device of claim 3.

24. The package of claim 21 wherein the implantable medical device is the device of claim 4.

25. The package of claim 21 wherein the implantable medical device is the device of claim 7.

26. An assembly comprising:
(a) a medical device implantable in the human eye;
(b) a package for storing the device during storage and shipping; wherein the medical device is the device of claim 1.

27. The assembly of claim 26 wherein the medical device is the device of claim 2.

28. The assembly of claim 26 wherein the medical device is the device of claim 3.

29. The assembly of claim 26 wherein the medical device is the device of claim 4.

30. The assembly of claim 26 wherein the medical device is the device of claim 7.

31. The assembly of claim 26 wherein the medical device is the device of claim 4

32. The assembly of claim 26 wherein the assembly is sterilized.

33. The assembly of claim 26 wherein the assembly is sterilized by gamma irradiation.

34. The assembly of claim 27 wherein the assembly is sterilized.

35. The assembly of claim 27 wherein the assembly is sterilized by gamma irradiation.

36. The assembly of claim 28 wherein the assembly is sterilized.

37. The assembly of claim 28 wherein the assembly is sterilized by gamma irradiation.

38. The assembly of claim 29 wherein the assembly is sterilized.

39. The assembly of claim 29 wherein the assembly is sterilized by gamma irradiation.

40. The assembly of claim 30 wherein the assembly is sterilized.

41. The assembly of claim 30 wherein the assembly is sterilized by gamma irradiation.
